(19) United States
(12) Patent Application Publication
(10) Pub. No.: US 2005/0027143 A1
(43) Pub. Date: Feb. 3, 2005

(54) Process for the preparation of beta-ionylideneacetaldehyde

(76) Inventors: Mohammad Salman, Gurgaon (IN); Purna Chandra Ray, New Delhi (IN); Suresh Jayachandra Babu, Gurgaon (IN); Naresh Kumar, Chandigarh (IN)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 10/487,554

(22) PCT Filed: Aug. 23, 2002
(86) PCT No.: PCT/IB02/03432

(30) Foreign Application Priority Data
Aug. 24, 2001 (IN) 880/DEL/01

(51) Int. Cl. C07C 45/45

(52) U.S. Cl. 568/447

(57) ABSTRACT

The present invention relates to an industrially advantageous process for the preparation of beta-ionylidencacetaldehyde of structural Formula I, which is a key intermediate for the synthesis of vitamin A and related compounds such as tretinoin and isotretinoin.

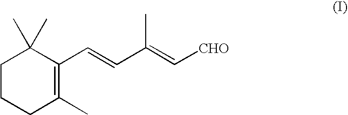

### FIELD OF THE INVENTION

The present invention relates to an industrially advantageous process for the preparation of β-ionylideneacetaldehyde of structural Formula I: 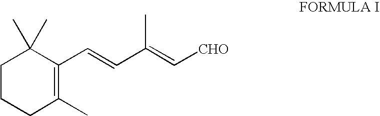
β-ionylideneacetaldehyde is a key intermediate in the synthesis of vitamin A and related compounds such as tretinoin and isotretinoin. These compounds have wide variety of biological activities e.g. isotretinoin inhibits sebaceous gland function and keratinization and are useful in the treatment of dermatological diseases like acne. Isotretinoin has also been evaluated for its anti-cancer activity.

### BACKGROUND OF THE INVENTION

The synthesis of β-ionylideneacetaldehyde utilizes β-ionone as the starting material. All the double bonds in β-ionylideneacetaldehyde have trans configuration and the major synthetic challenge has been to maintain the conjugated trans-polyene system in the molecule. The available synthetic approaches for β-ionylideneacetaldehyde are summarized below.

J. Am. Chem. Soc., 1955; 77: 4111 discloses the synthesis of the cis and trans ethyl β-ionylideneacetates using Reformatsky reaction. This approach involves the condensation of ethyl bromoacetate with β-ionone in the presence of zinc to give β-ionylideneacetate as a mixture of cis and trans in the ratio of 7:3, respectively. This ester, upon saponification and selective crystallization, gives trans β-ionylideneacetic acid In very poor (˜20%) yield. The acid intermediate is esterified and reduced using lithium aluminium hydride to give trans β-ionylidene ethanol; oxidation of alcohol intermediate finally affords the desired β-ionylideneacetaldehyde. Although this approach maintains the trans geometry at the C-9 bond but it is not commercially viable as it involves several steps and extremely poor over-all yield; selectivity of the C-9 double bond formation at Reformatsky stage in ethyl β-ionylideneacetate lowers the yield of the desired trans isomer, rendering the process uneconomical.

Bull. Chem. Japan, 1963, 1527 describes the synthesis of ethyl β-ionylideneacetate by means of a Wittig reaction using diethyl carboxymethylphosphonate prepared from triethyl phosphite and ethyl bromoacetate.

β-ionylideneacetate is synthesized by condensing the β-ionone with diethylcarboxymethylphosphonate in the presence of sodium amide in tetrahydrofuran. This acetate is reduced with lithium aluminium hydride in ether to give β-ionylidene ethanol, followed by its oxidation with manganese dioxide to give the desired β-ionylideneacetaldehyde. The oxidation is performed in petroleum ether at room temperature for 24 hours. This process is unacceptable on a commercial scale because the process requires maintaining the temperature (30° C.) for 24 hours. More importantly, we found that this process was not stereoselective; the ester, alcohol and the desired aldehyde were not 100% trans, rather a mixture of 9-cis and 9-trans isomers were obtained.

Gazz. Chem. 1973; 103: 117 discloses the synthesis of β-ionylideneacetaldehyde by the condensation of β-ionone with lithioacetonitrile (generated from n-butyl lithium and acetonitrile) to give β-ionylideneacetonitrile with almost 60%, trans selectively. After chromatographic purification, trans β-ionylideneacetonitrile is reduced with dilsobutylaluminium hydride (DIBAL) to afford β-ionylideneacetaldehyde which is further purified by chromatography. This process is not attractive for operation at commercial level, since it Involves column chromatography at the intermediate or penultimate stages of the preparation of β-ionylideneacetaldehyde. The trans selectivity of C-9 double bond in the preparation of β-ionylideneacetonitrile is poor and requires chromatographic purification before transformation to the aldehyde. The desired aldehyde also requires chromatographic purification, making this approach commercially difficult to implement.

In Chem. Pharm. Bull. 1994; 42(3): 757 discloses an improved process by improving the trans-selectivity at the C-9 in the above approach. The process involves the preparation of a tricarbonyl iron complex of β-ionone by reacting β-ionone with triiron dodecacarbonyl in benzene which is condensed with lithium acetonitrile in tetrahydrofuran at −70° C. to afford the nitrile compound. The nitrile intermediate is subjected to the oxidative decomplexation with cupric chloride, followed by DIBAL reduction to afford the desired trans β-ionylideneacetaldehyde. This approach is also not suitable from commercial point of view as it involves a number of steps to generate the trans β-ionylidene-acetaldehyde, and also requires the use of expensive triiron dodecacarbonyl.

In view of the above drawbacks in the prior art processes, there is a need for the development of a simpler and efficient process for the preparation of β-ionyledineacetaldehyde with desired ratio of trans-isomer.

### SUMMARY OF THE INVENTION

The present invention overcomes the problems associated with the prior art and provides a simpler way for obtaining β-ionylideneacetaldehyde in less time and in fewer steps. The invention also avoids the tedious and cumbersome purification process of column chromatography, usage of expensive chemicals, solvents and has obvious benefits with respect to economics and convenience to operate on a commercial scale. Thus, the present invention provides a more commercially viable process for the preparation of pharmaceutically important compounds such as isotretinoin, tretinoin, vitamin A, etc.

Accordingly, the present invention provides a process for the synthesis of β-ionylideneacetaldehyde Formula I which comprises: 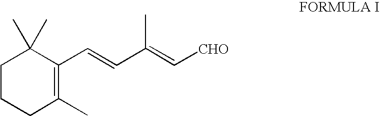
(a) condensing β-ionone of structural Formula II with triethyl phosphonoacetate of structural Formula III in the presence of sodium amide and toluene to obtain ethyl β-ionylideneacetate of Formula IV, 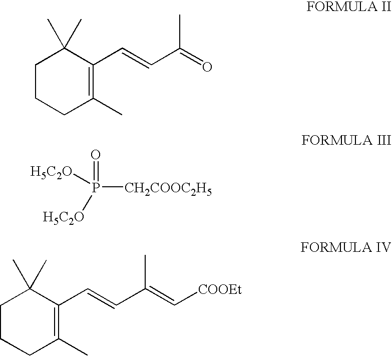
(b) reducing the ester of Formula IV to β-ionylidenealcohol of Formula V in the presence of organic solvent selected from hexane, tetrahydrofuran, toluene, xylene, and mixture(s) thereof, and 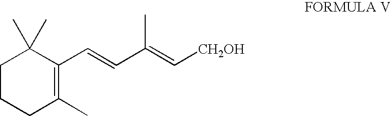
(c) oxidizing the alcohol of Formula V In situ with manganese dioxide at 60-70° C. for about 2 to 4 hours to obtain trans β-ionylidene acetaldehyde of structural formula I.

β-ionylideneacetaldehyde, so obtained may be converted into Vitamin A and related compounds such as tretinoin and isotretinoin by methods known in the art.

The process of condensation in step (a) is achieved by the reaction of β-ionone of Formula II with triethyl phosphonoacetate of Formula III in the presence of sodium amide and an inert organic solvent such as toluene. After a suitable aqueous work up, the ethyl β-ionylideneacetate of Formula IV is obtained as a mixture of 9-cis and 9-trans isomers in the ratio of 1:7.

The process of reduction in step (b) involves the reaction of ester of Formula IV with a reducing agent in organic solvent selected from hexane, tetrahydrofuran, toluene, xylene, and mixture (s) thereof at room temperature. The reducing agent used is selected from the group consisting of lithium aluminium hydride, sodium bis (2-methoxyethoxy) aluminium hydride (Red-Al) and diisobutyl aluminium hydride (DIBAL).

The alcohol of Formula V obtained after aqueous acidic work up is oxidized in situ by reacting with manganese dioxide at 60-70° C. for 2 to 4 hours. After the reaction is completed, the desired trans β-ionylideneacetaldehyde is obtained in more than 90% yield having less than 5% of 9-cis isomer.

Suitable aqueous work up involves the extraction with organic solvents. Any organic solvent may be used for extraction and such solvents are known to a person of ordinary skill In the art and include both water immisible and partially miscible solvent such as chloroform, methylene chloride, 1,2-dichloroethane, hexanes, cyclohexanes, toluene, methyl acetate, ethyl acetate, and the like.

Methods known in the art may be used with the process of this invention to enhance any aspect of this process for example, the product obtained may be further purified by recrystallization from solvent(s).

### DETAILED DESCRIPTION OF THE INVENTION

In the following section preferred embodiments are described by way of example to illustrate the process of the invention. However, these are not intended in any way to limit the scope of the present invention.

### EXAMPLE

### Preparation of β-ionylideneacetaldehyde (I)

Step a) Preparation of ethyl β-ionylideneacetate (IV)

A solution of triethyl phosphonoacetate (1.40 kg) in toluene (1 litre) was added at about 40° C. with stirring to a mixture of sodium amide (0.236 kg) and toluene (6.5 litre) under nitrogen atmosphere. The reaction mixture was stirred at 40-45° C. for six hours, it was then cooled to 0-5° C., and a solution of β-ionone (1 kg) in toluene (1.5 litre) was slowly added at 0°-10° C. The reaction mixture was stirred at 65° C. for 15 hours and cooled to 20-25° C. Water (4 litre) was added to the reaction mixture followed by stirring for another 15 minutes. The toluene layer was separated and distilled under vacuum at 60-80° C. to yield the titled compound of Formula IV in 87% yield as a mixture of 9-cis and 9-trans isomers in the ratio of 1:7.

Step b) Preparation of β-ionylidene ethanol (V)

Lithium aluminium hydride (0.11 kg) was added with stirring to the reaction mixture containing hexanes and tetrahydrofuran (4.5:1 litre) under nitrogen atmorphere. The reaction mixture was stirred for 30 minutes, cooled to 5-10° C., a solution of the ethyl β-ionylideneacetate (1 kg) in hexane was added slowly at 10-12° C. with stirring. The reaction mixture was further stirred for one hour at the same temperature, then cooled to 0-2° C., and sulfuric acid (0.88 litre) was added very slowly with stirring at 0-10° C. over a period of 40-50 minutes. The reaction mixture was stirred at 10-12° C. for one hour. It was then filtered to remove the inorganic solids, the cake was washed with hexanes. The combined organic layer was then washed with water and used as such in the next step.

Step c) Preparation of β-ionylideneacetaldehyde (I)

Manganese dioxide (3 kg) was added to the solution of β-ionylidene alcohol obtained in the previous step with stirring at room temperature. The reaction mixture was then refluxed at 60° C. for three hours and then filtered. The filter cake was washed with hexane. The combined hexane layer was distilled under vacuum to yield the titled compound of Formula I in 93% yield having less than 5% of 9-cis isomer.

While the present invention has been described in terms of its specific embodiments, certain modifications and equivalents will be apparent to those skilled in the art and are intended to be included within the scope of the present invention.

1. A process for the synthesis of β-ionylideneacetaldehyde of structural Formula I which comprises: 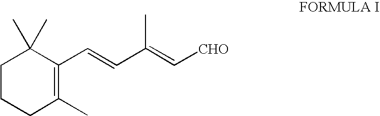
(a) condensing β-ionone of structural Formula II with triethyl phosphonoacetate of structural Formula III to obtain ethyl β-ionylideneacetate of structural Formula IV, 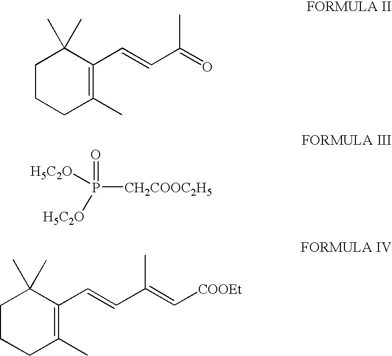
(b) reducing the ester of Formula IV with a reducing agent to β-ionylidene alcohol of Formula V in the presence of organic solvent, and 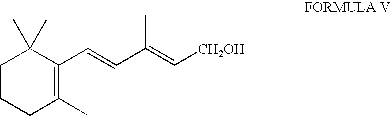
(c) oxidizing the alcohol of Formula V in situ with manganese dioxide at 60-70° C. for about 2 to 4 hours to obtain β-ionylideneacetaldehyde of structural Formula I.

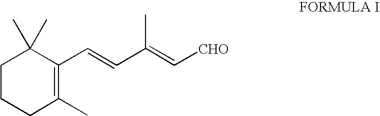

2. The process of claim I wherein the condensation of β-ionone with triethyl phosphonoacetate is carried out in the presence of sodium and toluene.

3. The process of claim 1 wherein the reducing agent is selected from the group consisting of lithium aluminium hydride, sodium bis (2-methoxyethoxy) aluminium hydride (Red-Al), and diisobutyl aluminium hydride (DIBAL).

4. The process of claim 3 wherein the reducing agent is lithium aluminium hydride (LAH).

5. The process of claim 1 wherein the organic solvent is selected from the group consisting of hexane, tetrahydrofuran, toluene, xylene, and mixture(s) thereof.

6. The process of claim 1 wherein the trans β-ionylideneacetaldehyde has less than 5% of the 9-cis isomer.

7. A process for the synthesis of ethyl β-ionylideneacetate of structural Formula IV 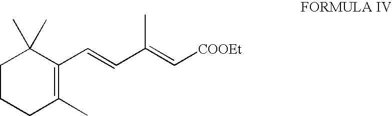
which comprises the condensing β-ionone of structural Formula II 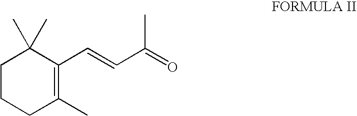
with triethyl phosphonoacetate of structural Formula III 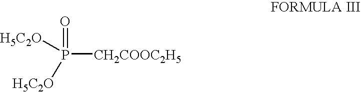
in the presence of sodium amide.

8. A process for the preparation of β-ionylidene alcohol of Formula V 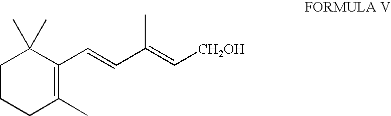
which comprises reducing the ester of Formula IV 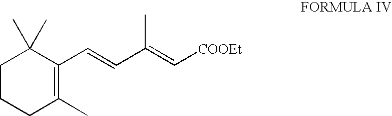
with a reducing agent in the presence of organic solvent.

9. The process of claim 8 wherein the organic solvent is selected from the group consisting of hexane, tetrahydrofuran, toluene, xylene, and mixture(s) thereof.

10. The process of claim 8 wherein the reducing agent is selected from the group consisting of lithium aluminium hydride, sodium bis (2-methoxyethoxy) aluminium hydride (Red-Al), and diisobutyl aluminium hydride (DIBAL).

11. A process for the preparation of trans β-ionylideneacetaldehyde of Formula I 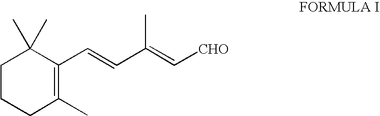
which comprises oxidizing the alcohol of Formula V 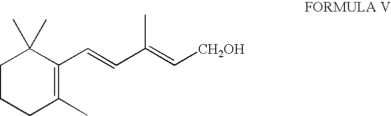
with manganese dioxide at 60-70° C. for about 2 to 4 hours.

12. The process according to claim 1 wherein the β-ionolideneacetaldehyde is converted into tretinoin.

13. The process according to claim 1 wherein the β-ionolideneacetaldehyde is converted into isotretinoin.

14. The process according to claim 1 wherein the β-ionolideneacetaldehyde is converted into Vitamin A.
